(19) United States
(12) Patent Application Publication
(10) Pub. No.: US 2005/0146070 A1
(43) Pub. Date: Jul. 7, 2005

(54) Meta lback or mesh crosslinking

(75) Inventors: Orhun K Muratoglu, Cambridge, MA (US); William H Harris, Belmont, MA (US)

Correspondence Address: HELLER EHRMAN WHITE & MCAULIFFE LLP, 1717 RHODE ISLAND AVE, NW, WASHINGTON, DC, 20036-3001, US

(73) Assignee: Massachusetts General Hospital, Bostan, MA (US)

(21) Appl. No.: 10/518,593

(22) PCT Filed: Jun. 10, 2003
(86) PCT No.: PCT/US03/18053

Related U.S. Application Data
(60) Provisional application No. 60/390,120, filed on Jun. 21, 2002.
(60) Provisional application No. 60/424,709, filed on Nov. 8, 2002.

(51) Int. Cl. B29C 43/18; B29C 43/52; A61F 2/30; A61F 2/34

(52) U.S. Cl. 264/85; 264/273; 264/488; 623/23.58; 623/22.33; 623/23.54

(57) ABSTRACT

The present invention relates to medical implants that comprise crosslinked polymeric material (such as UHMWPE) that is in contact with another piece (such as a metallic mesh or back, a non-metallic mesh or back, a tibial tray, a patella tray, or an acetabular shell), thereby forming an interface. Also disclosed herein are the methods of manufacturing and sterilizing such medical devices and materials used therein.

This application claims priority to U.S. Ser. No. 60/390,120, filed Jun. 21, 2002, and U.S. Ser. No. 60/424,709, filed Nov. 08, 2002, the entireties of which are hereby incorporated by reference.

### FIELD OF THE INVENTION

The present invention relates to a medical implant that comprises polymeric material that is in contact (which includes close proximity and touching) with another piece (such as a metallic mesh or back, a non-metallic mesh or back, a tibial tray, a patella tray, or an acetabular shell), thereby forming an interface. Methods of manufacturing and sterilizing such devices and materials used therein also are provided.

### BACKGROUND OF THE INVENTION

Increased crosslink density in polyethylene is desired in bearing surface applications for joint arthroplasty because it significantly increases the wear resistance of this material. The preferred method of crosslinking is by exposing the polyethylene to ionizing radiation. However, ionizing radiation, in addition to crosslinking, also will generate residual free radicals, which are the precursors of oxidation-induced embrittlement. This is known to adversely affect in vivo device performance. Therefore, it is desirable to reduce the concentration of residual free radicals, preferably to undetectable levels, following irradiation so as to avoid long-term oxidation.

Methods of irradiating polymers are described in U.S. Pat. No. 5,897,400. In general, this patent describes medical prosthesis formed, at least in part, of a melt-irradiated crosslinked high molecular weight polyethylene. The disclosed melt-irradiation process improves the wear resistance of the polymer, thus addressing the problem of severe adverse effects associated with the use of less wear resistant polymers. U.S. Pat. No. 5,897,400 describes, among other things, heating the polymers to or above the melting point, irradiating the polymer, and cooling the polymer.

International Application No. PCT/US97/02220 (WO 97/29793) also describes the irradiation of polymers that are useful in the orthopedic field. In this application, several methods of increasing the wear characteristics of polymers are described. The application describes, among other things, an irradiation procedure wherein the polymer is irradiated at room temperature or below. Following irradiation, the polymer can be heated to or above the melting temperature to remove any residual free radicals through the process of recombination. The application also describes another irradiation method in which the polymer is pre-heated to a temperature above room temperature, but below the melting temperature, and irradiated. Following irradiation, the polymer may be subsequently melted by heating it to the melting temperature or above to substantially eliminate any detectable free radicals via the process of recombination.

WO 97/29793 also describes methods of irradiating polymers in which the heat generated by the irradiation is sufficient to at least partially melt the polymer, and is described as “adiabatic” melting or heating. Adiabatic melting or heating refers to heating induced by radiation, which leads to an increase of the temperature of the polymer with substantially little loss of heat to the surroundings. The application describes an adiabatic melting method, among other things, in which the polymer is preheated to a temperature below the melting point, then irradiated with enough total dose and at a high enough dose rate to at least partially melt the polymer crystals. Subsequent to this warm-irradiation, the polymer also can be heated to or above the melting temperature such that any residual free radicals are eliminated. The application also describes another irradiation, adiabatic melting method that is similar to the method described above, except that the polymer is provided at room temperature or below.

It is important that crosslinked ultra-high molecular weight polyethylene (UHMWPE) based medical devices are sterilized after they are packaged for in vivo use. There are several methods of sterilization that can be utilized for medical implants, such as the use of ethylene oxide, gas plasma, heat (autoclave), or radiation. However, applying heat to a packaged polymeric medical product can destroy either the integrity of the packaging material (particularly the seal, which is provided to prevent bacteria and other contaminants from entering the package after the sterilization step) or the product itself. Because ethylene oxide may adversely impact environmental and employee safety, gamma ray, x-ray or electron beam radiation has been utilized as a preferred means of sterilization. These types of radiation use a high-energy beam to destroy or inactivate bacteria, viruses, and other microbial species that contaminate the packaged medical products.

However, it has been recognized that regardless of the radiation type, the high-energy beam causes generation of free radicals in polymers during irradiation. It also has been recognized that the amount of free radicals generated is dependent upon the radiation dose received by the polymers and that the distribution of free radicals in the polymeric implant depends upon the geometry of the component, the type of polymer, the dose rate, and the type of radiation.

Currently available methods of sterilization of medical devices containing polymeric materials include use of ethylene oxide (EtO) or gas plasma (GP). Although EtO and GP are successfully used in sterilizing certain polymeric implants, despite environmental and safety concerns, there are some implant designs where these gas sterilization methods will not work. These medical implants often contain factory-assembled pieces (usually a metallic or ceramic component) that are in close contact with the UHMWPE. In most cases, the interface is not accessible to the EtO gas or the GP. Therefore, these implants must be sterilized by gamma radiation, in air or in inert atmospheres. Yet these methods generate enough residual free radicals to adversely affect device performance in the long-term. With the recent introduction of radiation and thermal treatment of UHMWPE to improve its wear behavior and its long-term stability, gamma sterilization is no longer favored. In view of the limitations of the commercialized processes, new approaches are needed that will provide an alternative method of manufacturing crosslinked polymeric material (for example, UHMWPE)-based medical implants in configurations wherein the polymeric component is in close contact with another piece (such as another component, for example, a metallic or a non-metallic component).

The current invention, therefore, provides improved methods of making and sterilizing medical implants that contains highly cross-linked polymeric material, wherein the polymeric component (for example, UHMWPE) is in close contact with another piece (such as another component, for example, a metallic or a non-metallic component).

### SUMMARY OF THE INVENTION

The present invention relates generally to methods of manufacturing and sterilizing medical implants comprising a polymeric material, such as cross-linked ultra-high molecular weight polyethylene (UHMWPE), that is in contact with another piece (generally a metallic piece), thereby forming an interface, for example, an interlocking interface.

In one aspect, the invention provides methods of making a medical implant containing crosslinked polyethylene, for example, ultra-high molecular weight polyethylene (UHMWPE) that is in contact with another piece, thereby forming an interface, comprising the steps of: a) compression molding of polyethylene, including powder, flakes and particles to another piece, thereby forming an interlocked hybrid material; b) irradiating the hybrid material by ionizing radiation; and c) reducing free radicals in the crosslinked polyethylene by heating the hybrid material above the melting point of the crosslinked polyethylene. Polyethylene sheets also can be employed, but they may not result in the attainment of interlocking interfaces.

The metallic or non-metallic back or mesh as described, in one aspect of the invention, is shaped to serve as a fixation interface with the bone, through either bony growth or by bone cement, wherein the shapes are in the form of acetabular liner, tibial tray for total or unicompartmental knee implants, patella tray, glenoid component, ankle, elbow or finger component.

In another aspect, the invention includes methods of making a medical implant containing crosslinked polyethylene, for example, UHMWPE, that is in contact with another piece, thereby forming an interface, wherein the polyethylene, including powder, flakes and particles are compression molded to a metallic mesh, wherein the metallic mesh is shaped to serve as a fixation interface with the bone, through either bony growth or by bone cement, wherein the shapes are, for example, in the form of acetabular liner, tibial tray for total or unicompartmental knee implants, patella tray, glenoid component, ankle, elbow or finger component.

The polyethylene, as described herein, is in contact with another piece, thereby forming an interface, for example, an interlocking interface, wherein the interface is rendered substantially sterile.

In another aspect, the invention provides methods of making medical devices including bipolar hip replacements, tibial knee inserts with reinforcing metallic and polyethylene posts, and an implant that contains an interface that cannot be sterilized by a gas sterilization method.

In another aspect, the invention provides medical implants manufactured by the methods described herein.

In another aspect, the invention provides methods of: sterilizing a medical implant containing crosslinked polyethylene that is in contact with another piece, thereby forming an interface, wherein the methods comprise the steps of a) compression molding of polyethylene, such as resin powder, flakes and particles to another piece, thereby forming an interlocked hybrid material; b) irradiating the hybrid material by ionizing radiation; c) reducing free radicals in the crosslinked polyethylene by heating the hybrid material above the melting point of the crosslinked polyethylene; and d) sterilizing the medical implant with a gas.

Another aspect of the present invention includes methods of sterilization, wherein the implants are further sterilized by a gas, wherein the gas is ethylene oxide, gas plasma, or the other gas, wherein ethylene oxide, gas plasma, or the other gas, is used for gas sterilization.

In another aspect, the invention provides medical implants, comprising crosslinked polyethylene that is in contact with another piece, thereby forming an interface, made by processes comprising the steps of: a) compression molding of polyethylene, such as resin powder, flakes and particles to another piece, thereby forming an interlocked hybrid material; b) irradiating the hybrid material by ionizing radiation; and c) reducing free radicals in the crosslinked polyethylene by heating the hybrid material above the melting point of the crosslinked polyethylene.

In another aspect, the invention provides medical implants, comprising crosslinked polyethylene that is in contact with another piece, thereby forming an interface, made by processes comprising the steps of: a) compression molding of polyethylene, such as resin powder, flakes and particles to another piece, thereby forming an interlocked hybrid material; b) irradiating the hybrid material by ionizing radiation; and c) reducing free radicals in the crosslinked polyethylene by heating the hybrid material above the melting point of the crosslinked polyethylene.

In another aspect, the invention provides interfaces made by processes comprising the steps of: a) compression molding of polyethylene, such as resin powder, flakes and particles to another piece, thereby forming an interlocked hybrid material; b) irradiating the hybrid material by ionizing radiation; and c) reducing free radicals in the polyethylene by heating the hybrid material above the melting point of the polyethylene.

In another aspect, the invention provides interfaces made by processes comprising the steps of: a) compression molding of polyethylene to another piece, thereby forming an interlocked hybrid material; b) irradiating the hybrid material by ionizing radiation; and c) reducing free radicals in the polyethylene by heating the hybrid material above the melting point of the polyethylene.

In another aspect, the invention provides interfaces made by processes comprising the steps of: a) compression molding of polyethylene, such as resin powder, flakes and particles to another piece, thereby forming an interlocked hybrid material; and b) irradiating the hybrid material by ionizing radiation, wherein the interface is rendered substantially sterile.

In another aspect, the invention provides interfaces made by processes comprising the steps of: a) compression molding of polyethylene, such as resin powder, flakes and particles to another piece, thereby forming a mechanically interlocked hybrid material; and b) irradiating the hybrid material by ionizing radiation, wherein the interface is rendered substantially sterile.

In another aspect, the invention provides interfaces made by processes comprising the steps of: a) compression molding of polyethylene, such as resin powder, flakes and particles to another piece, thereby forming an interlocked hybrid material; b) irradiating the hybrid material by ionizing radiation; and c) reducing free radicals in the polyethylene by heating the hybrid material above the melting point of the polyethylene, wherein the interface is rendered substantially sterile.

In another aspect, the invention provides interfaces made by processes comprising the steps of: a) compression molding of polyethylene, such as resin-powder, flakes and particles to another piece, thereby forming an interlocked hybrid material; b) irradiating the hybrid material by ionizing radiation; and c) reducing free radicals in the polyethylene by heating the hybrid material above the melting point of the polyethylene, wherein the interface is rendered substantially sterile.

In another aspect, the invention provides acetabular assemblies comprising: a) polyethylene powder compression molded to another piece, thereby forming an interlocked hybrid material; b) a substantially sterile interface; and c) a metallic back.

In another aspect, the invention provides acetabular assemblies comprising: a) a polyethylene acetabular liner compression molded to another piece, thereby forming a mechanically interlocked hybrid material; b) a substantially sterile interface; and c) a metallic back.

Another aspect of the invention provides medical implants comprising crosslinked polyethylene having substantially no detectable free radicals; and at least one substantially sterile interface, for example, a substantially sterile mechanically interlocking interface.

Another aspect of the invention provides medical implants comprising crosslinked polyethylene having substantially no detectable free radicals; and a sterile mechanically interlocking interface, wherein polyethylene, such as resin powder, flakes and particles are compression molded to another piece, for example, a metallic mesh or back, a non-metallic mesh or back such as ceramic mesh or back, a tibial tray, a patella tray, or an acetabular shell.

The heating temperature of the compressed polyethylene or the hybrid components are above the melting point of the polyethylene, preferably above about 137° C. and the radiation dose at the melt is between about 25 kGy and about 1000 kGy. The radiation dose can be about 50 kGy, about 100 kGy, about 200 kGy, about 300 kGy, about 400 kGy, about 500 kGy, about 600 kGy, about 700 kGy, about 800 kGy, about 900 kGy, or about 1000 kGy.

In one aspect of the invention, the heating of the hybrid material above the melting point of the polyethylene is carried out in air, wherein the air contains between about 1% and about 22% oxygen. In a preferred aspect of the invention, the heating is carried out in air containing between about 2% and about 21% oxygen.

In another aspect, the heating of the hybrid material above the melting point of the polyethylene is carried out in an inert atmosphere, wherein the inert atmosphere contains gas selected from the group consisting of nitrogen, argon, helium, neon, or the like, or a combination thereof.

In another aspect, the heating of the hybrid material above the melting point of the polyethylene is carried out in a vacuum.

The counterface piece as described, in one aspect of the invention, comprises a metal or a non-metal, wherein the piece is a metallic mesh or back, a ceramic mesh or back, a tibial tray, a patella tray, or an acetabular shell, wherein the piece comprises a metallic mesh or back, a non-metallic mesh or back, an undercut, a recess or a combination thereof.

The interfaces as described in one aspect of the invention, comprise a metal-polymer, wherein the polymer is a polyolefin, wherein the polyolefin is selected from a group consisting of a low-density polyethylene, high-density polyethylene, linear low-density polyethylene, ultra-high molecular weight polyethylene (UHMWPE), or mixtures thereof In another aspect, the invention provides methods of sterilization, wherein the implant comprises medical devices selected from the group consisting of bipolar hip replacements, tibial knee inserts with reinforcing metallic and polyethylene posts, and an implant that contains an interface that cannot be readily sterilized by a gas sterilization method.

In one aspect of the invention, the ionizing radiation includes gamma or ionizing irradiation in air, wherein the air contains between about 1% and about 22% oxygen. In a preferred aspect of the invention, the radiation is carried out in air containing between about 2% and about 21% oxygen.

In another aspect, the ionizing radiation includes gamma or ionizing irradiation in an inert atmosphere, wherein the inert atmosphere contains gas selected from the group consisting of nitrogen, argon, helium, neon, or the like, or a combination thereof.

In another aspect, the ionizing radiation includes gamma or ionizing irradiation in a vacuum.

Following irradiation, the reduction of free radicals in the crosslinked polyethylene is achieved by heating the implants to above the melting temperature of the polyethylene, wherein the polyethylene can be in contact with a non-oxidizing medium, wherein the non-oxidizing medium is an inert gas or an inert fluid, wherein the medium is a polyunsaturated hydrocarbon selected from the group consisting of: acetylenic hydrocarbons such as acetylene; conjugated or unconjugated olefinic hydrocarbons such as butadiene and (meth)acrylate monomers; and sulphur monochloride with chloro-tri-fluoroethylene (CTFE) or acetylene.

In one aspect of the invention, the reduction of free radicals in crosslinked polyethylene is achieved in air, wherein the air contains between about 1% and about 22% oxygen. In a preferred aspect of the invention, the radiation is carried out in air containing between about 2% and about 21% oxygen.

In another aspect, the reduction of free radicals in crosslinked polyethylene is achieved in an inert atmosphere, wherein the inert atmosphere contains gas selected from the group consisting of nitrogen, argon, helium, neon, or the like, or a combination thereof.

In another aspect, the reduction of free radicals in crosslinked polyethylene is achieved in a vacuum.

In one aspect, the invention provides medical implants comprising crosslinked polyethylene having substantially no detectable free radicals; and a sterile interface such as a mechanically interlocking interface. The polyethylene of the implant as described herein is in contact with another piece, thereby forming an interface, wherein the polyethylene, such as resin powder, flakes and particles are compression molded to another piece, for example, a metal or a non-metal, thereby forming a mechanically interlocked hybrid material.

The implant as described herein is compression molded, wherein polyethylene, such as resin powder, flakes and particles are compression molded to another piece, for example, a metallic mesh or back, or a non-metallic mesh or back such as a ceramic mesh or back, a tibial tray, a patella tray, or an acetabular shell.

The implant as described herein comprises a metal-polymer interface, wherein the metal piece comprises a metallic mesh or back, or a non-metallic mesh or back such as a ceramic mesh or back, an undercut, a recess or a combination thereof, thereby forming a mechanically interlocked hybrid material.

### BRIEF DESCRIPTION OF THE DRAWINGS

FIG. 1 shows sequential steps in compression molding of polyethylene powder (10) to a metallic mesh (20).

FIG. 2 illustrates compression molding of polyethylene (60) on a metallic back (40) containing a metallic mesh (20) and a metallic backed Patella (70).

FIG. 3 shows compression molding of polyethylene (120) on a metal shell (100) containing metallic mesh (20).

FIG. 4 shows metallic tray (40) having mesh (20).

FIG. 5 shows metallic tray (40) having undercut (80).

FIG. 6 shows a schematic diagram of compression apparatus set up.

FIG. 7 depicts a typical mesh incorporated into UHMWPE pin by high temperature compression.

FIG. 8 illustrates a mesh incorporated into polyethylene by compression molding polyethylene resin powder.

FIG. 9 depicts an example of mesh retention in the polyethylene resin/mesh assembly following irradiation and subsequent melting, that is post-irradiation before melting.

FIG. 10 depicts an example of mesh retention in the polyethylene resin/mesh assembly following irradiation and subsequent melting, that is post-irradiation after melting.

### DETAILED DESCRIPTION OF THE INVENTION

The present invention relates generally to methods of manufacturing and sterilizing medical implants comprising a polymeric material, such as cross-linked ultra-high molecular weight polyethylene (UHMWPE), that is in contact with another piece (for example, a metallic or non-metallic component), thereby forming an interlocking interface.

One aspect of the invention relates to the following processing steps to fabricate medical devices made out of highly crosslinked UHMWPE and containing metallic pieces such as bipolar hip replacements, tibial knee inserts with reinforcing metallic and polyethylene posts, and for any implant that contains a surface that cannot be readily sterilized by a gas sterilization method.

According to one aspect of the invention, the UHMWPE component of a medical implant is in close contact with another material (such as a metallic mesh or back, a non-metallic mesh or back, a tibial tray, a patella tray, or an acetabular shell), wherein polyethylene, such as resin powder, flakes and particles are directly compression molded to these counterfaces. For example, a polyethylene tibial insert is manufactured by direct compression molding of polyethylene resin powder to a tibial tray, to a metallic mesh or back or to a non-metallic mesh or back. In the latter case, the mesh is shaped to serve as a fixation interface with the bone, through either bony in-growth or the use of an adhesive, such as PMMA bone cement. These shapes are of various forms including, acetabular liner, tibial tray for total or unicompartmental knee implants, patella tray, and glenoid component, ankle, elbow or finger component. One aspect of the invention relates to mechanical interlocking of the molded polyethylene with the other piece(s), for example, a metallic or a non-metallic piece, that makes up part of the implant.

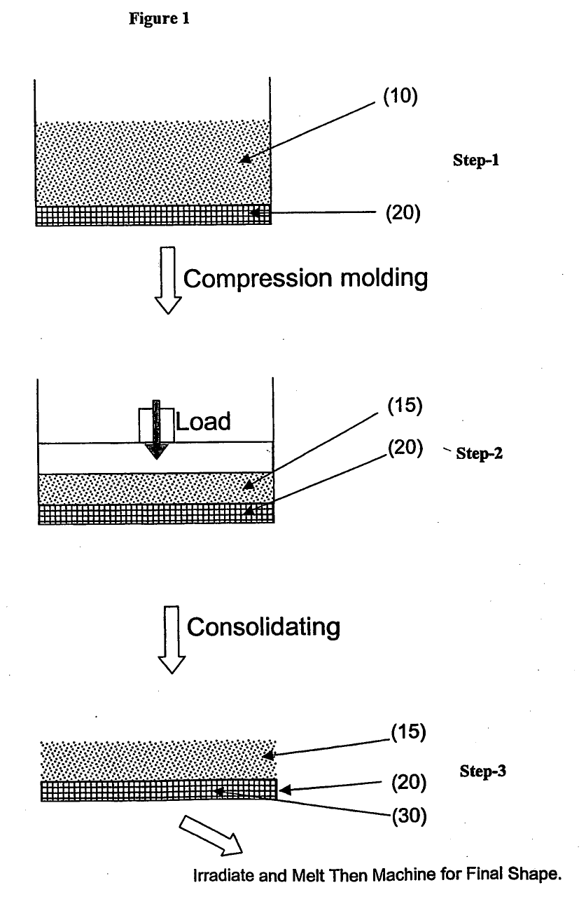

Another aspect of the invention provides methods for achieving mechanically interlocked medical devices. For example, a mesh-like surface on the other side facing the polyethylene powder is compression molded and the consolidated polyethylene takes a shape at the interface that penetrates into the mesh. The mesh surface on the other side can be continuous through the interface or intermittent (see FIG. 1).

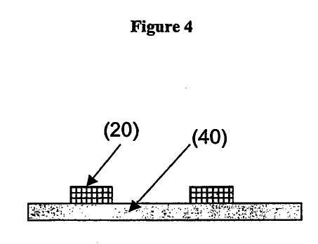

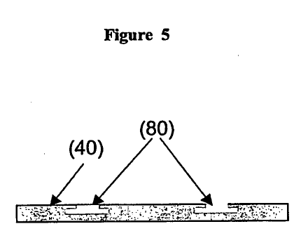

Another aspect of the invention includes surfaces with geometries, for example, undercuts, grooves (see FIGS. 4-5), or the like that allows the polyethylene, such as resin powder, flakes and particles to penetrate, consolidate and take the shape of the surface such that the mechanical interlocking achieved allows for a strong interface (see FIG. 4).

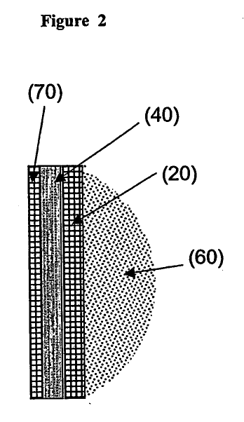

Another aspect of the invention provides combination of interface surfaces, for example, a mesh and macro-geometries are combined to tailor an interface of preferred strength (see FIG. 2).

The interface geometry is crucial in that polyethylene assumes the geometry as its consolidated shape. UHMWPE has a remarkable property of ‘shape memory’ due to its very high molecular weight that results in a high density of physical entanglements. Following consolidation, plastic deformation introduces a permanent shape change, which is almost completely reversed by melting. This recovery of the original consolidated shape is due to the ‘shape memory’, which is triggered by melting.

Another aspect of the invention provides that following the direct compression moldings of the polyethylene to the counterface with the mechanical interlock, the hybrid component is irradiated using ionizing radiation to a desired dose level, for example, about 25 kGy to about 1000 kGy, preferably between about 50 kGy and about 100 kGy. In accordance with aspects of the invention, the invention discloses that while the irradiation crosslinks the polymer, it also sterilizes the interfaces, that is the close contact between the polyethylene and the counterface. Another aspect of the invention discloses that the irradiation step generates residual free radicals and therefore, a melting step is introduced thereafter to quench the residual free radicals. Since the polyethylene is consolidated into the shape of the interface, thereby setting a ‘shape memory’ of the polymer, the polyethylene does not separate from the counterface.

In another aspect of the invention, there are provided methods of crosslinking polyethylene, to create a UHMWPE-based medical device, wherein the device is immersed in a non-oxidizing medium such as inert gas or inert fluid, wherein the medium is heated to above the melting point of the irradiated polyethylene, for example, UHMWPE (above about 137° C.) to eliminate the crystalline matter and to allow the recombination/elimination of the residual free radicals. Because the shape memory of the compression molded polymer is set at the mechanically interlocked interface and that memory is strengthened by the crosslinking step, there is no significant separation at the interface between the polyethylene and the counterface.

Another aspect of the invention provides that following the above steps of free radical elimination, the interface between the metal and the polymer become sterile due to the high irradiation dose level used during irradiation. When there is substantial oxidation on the outside surface of the polyethylene induced during the free radical elimination step or irradiation step, the device surface can be further machined to remove the oxidized surface layer. In another aspect, the invention provides that in the case of a post-melting machining of an implant, the melting step is carried out in the presence of an inert gas.

Another aspect of the invention includes methods of sterilization of the fabricated device, wherein the device is further sterilized with ethylene oxide, gas plasma, or the other gases, when the interface is sterile but the rest of the component is not.

The term “compression molding” as referred herein related generally to what is known in the art and specifically relates to molding applicable in polyethylene-based devices, for example, medical implants wherein polyethylene in any physical state, including powder form, is compressed to solid support, for example, a metallic back, metallic mesh, or metal surface containing grooves, undercuts, or cutouts. The compression molding also includes high temperature compression molding of polyethylene at various states, including resin powder, flakes and particles, to make a component of a medical implant, for example, a tibial insert, an acetabular liner, a glenoid liner, a patella, or an unicompartmental insert, to the counterface.

The term “mechanically interlocked” refers generally to interlocking of polyethylene and the counterface, that are produced by various methods, including compression molding, heat and irradiation, thereby forming an interlocking interface, resulting into a ‘shape memory’ of the interlocked polyethylene. Components of a device having such an interlocking interface can be referred to as a “hybrid material”. Medical implants having such a hybrid material, contain a substantially sterile interface.

The term “substantially sterile” refers to a condition of an object, for example, an interface or a hybrid material or a medical implant containing interface(s), wherein the interface is sufficiently sterile to not result in infection or require revision surgery.

“Metallic mesh” refers to a porous metallic surface of various pore sizes, for example, 0.1-3 mm. The porous surface can be obtained through several different methods, for example, sintering of metallic powder with a binder that is subsequently removed to leave behind a porous surface; sintering of short metallic fibers of diameter 0.1-3 mm; or sintering of different size metallic meshes on top of each other to provide an open continuous pore structure.

“Bone cement” refers to what is known in the art as an adhesive used in bonding medical devices to bone. Typically, bone cement is made out of polymethylmetacrylate (PEMMA).

“High temperature compression molding” refers to the compression molding of polyethylene in any form, for example, resin powder, flakes or particles, to impart new geometry under pressure and temperature. During the high temperature (above the melting point of polyethylene) compression molding, polyethylene is heated to above its melting point pressurized into a mold of desired shape and allowed to cool down under pressure-to maintain a desired shape.

“Shape memory” refers to what is known in the art as the property of polyethylene, for example, an UHMWPE, that attains a preferred low energy shape when melted. The preferred low energy shape is achieved when the resin powder is consolidated through compression molding.

The phrase “substantially no detectable residual free radicals” refers to a state of a polyethylene component, wherein enough free radicals are eliminated to avoid oxidative degradation, which can be evaluated by electron spin resonance (ESR). The lowest level of free radicals detectable with state-of-the-art instruments is about 10¹⁴ spins/gram and thus the term “detectable” refers to a detection limit of 10¹⁴ spins/gram by ESR.

The terms “about” or “approximately” in the context of numerical values and ranges refers to values or ranges that approximate or are close to the recited values or ranges such that the invention can perform as intended, such as having a desired degree of crosslinking and/or a desired lack of free radicals, as is apparent to the skilled person from the teachings contained herein. This is due, at least in par, to the varying properties of polymer compositions. Thus these terms encompass values beyond those resulting from systematic error.

Polymeric Material:

Ultra-high molecular weight polyethylene (UHMWPE) refers to linear non-branched chains of ethylene having molecular weights in excess of about 500,000, preferably above about 1,000,000, and more preferably above about 2,000,000. Often the molecular weights can reach about 8,000,000 or more. By initial average molecular weight is meant the average molecular weight of the UHMWPE starting material, prior to any irradiation. See U.S. Pat. No. 5,879,400, PCT/US99/16070, filed on Jul. 16, 1999, and PCT/US97/02220, filed Feb. 11, 1997, the entirety of which are hereby incorporated by reference.

The products and processes of this invention also apply to various types of polymeric materials, for example, a polyolefin, including high-density-polyethylene, low-density-polyethylene, linear-low-density-polyethylene, ultra-high molecular weight polyethylene (UHMWPE), or mixtures thereof. Polymeric materials, as used herein, also applies to polyethylene of various forms, for example, resin powder, flakes and particles.

Sterilization Steps:

The present invention relates to a method of sterilizing medical implants comprising a polymeric material, such as cross-linked UHMWPE, that is in contact with another piece, thereby forming an interface, comprising the steps of a) sterilizing an interface by ionizing radiation; b) reducing free radicals in the UHMWPE under inert conditions; c) heating the medium to above the melting point of the irradiated UHMWPE (above about 137° C.) to eliminate the crystalline matter and allow for the recombination/elimination of the residual free radicals and d) sterilizing the medical implant with a gas.

UHMWPE can be cross-linked by a variety of approaches, including those employing cross-linking chemicals (such as peroxides and/or silanes) and/or irradiation. Preferred approaches for cross-linking employ irradiation. Crossed linked UHMWPE can be obtained according to the teachings of U.S. Pat. No. 5,879,400 and PCT/US97/02220.

Interface:

The term interface in this invention is defined as the niche in medical devices formed when an implant is in a configuration where the UHMWPE is in contact with another piece (such as a metallic or a non-metallic component), which forms an interface between the polymer and the metal or another polymeric material. For example, interfaces of polymer-polymer or polymer-metal in medical prosthesis such as, orthopedic joints and bone replacement parts, e.g., hip, knee, elbow or ankle replacements.

Medical implants containing factory-assembled pieces that are in close contact with the UHMWPE form interfaces. In most cases, the interfaces are not readily accessible to EtO gas or the GP during a gas sterilization process.

Irradiation:

In one aspect of the invention, the type of radiation, preferably ionizing, is used. According to another aspect of the invention, a dose of ionizing radiation ranging from about 25 kGy to about 1000 kGy, preferably between about 50 kGy and about 100 kGy is used. The radiation dose can be about 50 kGy, about 100 kGy, about 200 kGy, about 300 kGy, about 400 kGy, about 500 kGy, about 600 kGy, about 700 kGy, about 800 kGy, about 900 kGy, or about 1000 kGy, or any integer therebetween. These types of radiation, including gamma and/or electron beam, kills or inactivates bacteria, viruses, or other microbial agents potentially contaminating medical implants, including the interfaces, thereby achieving product sterility: The irradiation, which may be electron or gamma irradiation, in accordance with the present invention is carried out in air atmosphere containing oxygen, wherein the oxygen concentration in the atmosphere is at least 1%, 2%, 4%, or up to about 22%, or any integer thereabout or therebetween. In another aspect, the irradiation is carried out in an inert atmosphere, wherein the atmosphere contains gas selected from the group consisting of nitrogen, argon, helium, neon, or the like, or a combination thereof. The irradiation also can be carried out in a vacuum.

In accordance with a preferred feature of this invention, the irradiation may be carried-out in a sensitizing atmosphere. This may comprise a gaseous substance which is of sufficiently small molecular size to diffuse into the polymer and which, on irradiation, acts as a polyfunctional grafting moiety. Examples include substituted or unsubstituted polyunsaturated hydrocarbons; for example, acetylenic hydrocarbons such as acetylene; conjugated or unconjugated olefinic hydrocarbons such as butadiene and (meth)acrylate monomers; sulphur monochloride, with chloro-tri-fluoroethylene (CTFE) or acetylene being particularly-preferred. By “gaseous” is meant herein that the sensitizing atmosphere is in the gas phase, either above or below its critical temperature, at the irradiation temperature.

Metal Piece:

In accordance with the invention, the piece forming an interface with polymeric material is, for example, a metal. The metal piece in functional relation with polyethylene, according to the present invention, can be made of a cobalt chrome alloy, stainless steel, titanium, titanium alloy or nickel cobalt alloy, for example.

Non-Metallic Piece:

In accordance with the invention, the piece forming an interface with polymeric material is, for example, a non-metal. The non-metal piece in functional relation with polyethylene, according to the present invention, can be made of ceramic material, for example.

Inert Atmosphere:

The term “inert atmosphere” refers to an environment having no more than 1% oxygen and more preferably, an oxidant-free condition that allows free radicals in polymeric materials to form cross links without oxidation during a process of sterilization. An inert atmosphere is used to avoid O₂, which would otherwise oxidize the medical device comprising a polymeric material, such as UHMWPE. Inert atmospheric conditions such as nitrogen, argon, helium, neon, or vacuum are used for sterilizing polymeric medical implants by ionizing radiation.

Inert atmospheric conditions such as nitrogen, argon, helium, neon, or vacuum are also used for sterilizing interfaces of polymeric-metallic and/or polymeric-polymeric in medical implants by ionizing radiation.

Vacuum:

The term “vacuum” refers to an environment having no appreciable amount of gas that allows free radicals in polymeric materials to form cross links without oxidation during a process of sterilization. An vacuum is used to avoid O₂, which would otherwise oxidize the medical device comprising a polymeric material, such as UHMWPE. A vacuum condition can be used for sterilizing polymeric medical implants by ionizing radiation.

A vacuum condition can be created using a commercially available vacuum pump. A vacuum condition also can be used when sterilizing interfaces of polymeric-metallic and/or polymeric-polymeric in medical implants by ionizing radiation.

Residual Free Radicals:

“Residual free radicals” refers to free radicals that are generated when a polymer is exposed to ionizing radiation such as gamma or e-beam irradiation. While some of the free radicals recombine with each other to from crosslinks, some become trapped in crystalline domains. The trapped free radicals are also known as residual free radicals.

According to one aspect of the invention, the levels of residual free radicals in the polymer generated during an ionizing radiation (such as gamma or electron beam) is preferably determined using electron spin resonance and treated appropriately to reduce free radicals.

Heating Process:

One aspect of the present invention discloses a process of reducing free radicals in polymeric component of a medical implant during the manufacturing process by heating for a time period depending on the melting temperature of the polymeric material. For example, the preferred temperature is about 137° C. or more.

Another aspect of the invention discloses a heating step that is carried in the air, in an atmosphere, containing oxygen, wherein the oxygen concentration is at least 1%, 2%,. 4%, or up to about 22%, or any integer thereabout or therebetween. In-another aspect, the invention discloses a heating step that is carried while the implant is in contact with an inert atmosphere, wherein the inert atmosphere contains gas selected from the group consisting of nitrogen, argon, helium, neon, or the like, or a combination thereof. In another aspect, the invention discloses a heating step that is carried while the implant is in contact with a non-oxidizing medium, such as a fluid medium, wherein the medium contains no more than about 1% oxygen. in another aspect, the invention discloses a heating step that is carried while the implant is in a vacuum.

In another aspect of this invention, there is described the heating method of implants to reduce residual free radicals. The medical device comprising a polymeric raw material, such as UHMWPE, is generally heated to a temperature of about 137° C. or more. The medical device is kept heated in the inert medium until the concentration of the residual free radicals is reduced to acceptable levels as measured by electron spin resonance. It is preferred that the concentration of the residual free radicals is at or below the detection limit of electron spin resonance.

Soaking Process:

The present invention also provides conditions of treatment of reducing residual free radicals by soaking the implant in a sensitizing atmosphere such as immersing the implant into acetylene or another sensitizing gas which can be pressured into the implant. In this process, the sensitizing gas diffuses in and reacts with residual free radicals forming additional cross-links. The invention further relates to a process, if necessary, to accelerate the diffusion of the sensitizing gas by increasing temperature of the chamber that holds the implant and the gas.

The term “contacted” in this context includes close physical proximity with or touching such that the sensitizing agent can perform its intended function. Preferably, a polyethylene composition or preform is sufficiently contacted such that it is soaked in the sensitizing agent, which ensures that the contact is sufficient. Soaking is defined as placing the sample in a specific environment for a sufficient period of time at an appropriate temperature. The environment include a sensitizing gas, such as acetylene, ethylene, or a similar gas or mixture of gases, or a sensitizing liquid, for example, a diene. The environment is heated to a temperature ranging from room temperature to a temperature above the melting point of the polymeric material. The contact period ranges from at least about 1 minute to several weeks and the duration depending on the temperature of the environment. In one aspect, the contact time period at room temperature is about 24 hours to about 48 hours and preferably about 24 hours.

A “sensitizing environment” refers to a mixture of gases and/or liquids (at room temperature) that contain sensitizing gaseous and/or liquid component(s) that can react with residual free radicals to assist in the recombination and elimination of the residual free radicals. The gases may be acetylene, chloro-trifluoro ethylene (CTFE), ethylene, or like. The gases or the mixtures of gases thereof may contain noble gases such as nitrogen, argon, neon and like. Other gases such as, carbon dioxide or carbon monoxide may also be present in the mixture. In applications where the surface of a treated material is machined away during the device manufacture, the gas blend could also contain oxidizing gases such as oxygen. The sensitizing environment can be dienes with different number of carbons, or mixtures of liquids and/or gases thereof. An example of a sensitizing liquid component is octadiene or other dienes, which can be mixed with other sensitizing liquids and/or non-sensitizing liquids such as a hexane or a heptane. A sensitizing environment can include a sensitizing gas, such as acetylene, ethylene, or a similar gas or mixture of gases, or a sensitizing liquid, for example, a diene. The environment is heated to a temperature ranging from room temperature to a temperature above the melting point of the polymeric material.

The invention is further described by the following examples, which do not limit the invention in any manner.

### EXAMPLES

### Example 1

### Compression Molding, Mechanical Interlocks in Medical Devices, and Irradiation of Hybrid Components

FIG. 1 diagrams sequential steps in compression molding of polyethylene powder to a metallic mesh. FIG. 1 Step-1 shows a mesh-like surface (20) on the other side facing the polyethylene powder (10) is compression molded. Step-2 shows the consolidated polyethylene (15) taking a shape at the interface that penetrates into the mesh and Step-3 depicting consolidated polyethylene partially penetrating the metallic mesh and forming a hybrid component (30) with an interlocking interface. The mesh surface on the other side is continuous through the interface or intermittent. Following compression molding, the hybrid component is irradiated, melted and then machined for final shape.

Polyethylene resin powder was placed on top of a metallic mesh as shown in FIG. 1 Step-1. These were then placed in a mold as shown in Step-2, heated to above the melting point and consolidated under pressure. Subsequent to pressing, the polyethylene was allowed to cool down to room temperature under pressure. The consolidated polyethylene was then removed from the mold to be irradiated to crosslink the polyethylene and melted to reduce the concentration of residual free radicals to an undetectable level. A final implant shape was then machined and the implant was sterilized with EtO, gas plasma or autoclaved. Extrusion of polyethylene on the other side of the mesh can be avoided by either having a solid layer of thin metal half-way through the mesh or by filling the empty pores on the backside with a space filling material that can be removed subsequent to the consolidation.

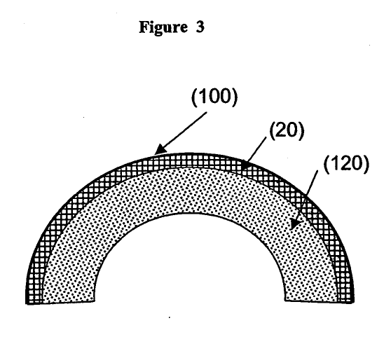

Referring to FIGS. 2-5, the diagram in FIG. 2 shows an example of a compression molded polyethylene (60) to a metallic back (40) containing metallic mesh (20) and a metallic backed Patella (70). FIG. 3 shows an example of a compression molded of polyethylene (120) to a metal shell (100) containing metallic mesh (20). FIGS. 4 and 5 show examples of metal trays (40) containing metallic mesh (20) and undercuts or recess (80), respectively, to secure compression molded polyethylene.

A surface with geometries, for example, mesh, recess, undercuts, grooves(see FIGS. 4 and 5), or the like that allowed the polyethylene resin powder to penetrate, consolidate and take the shape of the surface such that the mechanical interlocking is achieved and resulted into a hybrid component (30) having strong interlocking interface (see FIG. 1).

As shown in FIG. 2, polyethylene resin powder was consolidated to a mesh surface that has a solid metal backing for the manufacture of a patellar implant. The consolidated group was irradiated to crosslink the polyethylene and to sterilize the polyethylene/metallic mesh interface. Subsequently, the implant was melted to reduce the concentration of residual free radicals. The articulating surface of the implant was then machined to the desired geometry. Finally the implant was sterilized using a gas (ethylene oxide, gas plasma, or the other gas).

FIG. 3 shows the same as in FIG. 2, however, polyethylene resin powder was consolidated to a mesh surface in a different geometry.

FIGS. 4 and 5 depict two alternative counterface interlocking geometries. FIG. 4 shows an intermittent metallic mesh on a solid metallic back, wherein the mesh acts as the interlocking counterface. The intermittent mesh on a solid metal backing can be used in a variety of geometries such as a tibial knee insert, bipolar acetabular component, acetabular component, patellar components, or shoulder glenoid. FIG. 5 shows an undercut geometry into which polyethylene provides interfacial strength between the polyethylene and the metallic back. The shape and dimensions of the undercut can be varied to improve the interface strength. This type of interface geometry can be used in a variety of flat, concave, or convex interface geometries in a variety of implants.

As shown in FIGS. 2, 3, 4 and 5, the consolidated polyethylene can penetrate the full thickness of the metallic mesh. However, sufficient interface strength can be achieved even with partial penetration.

FIG. 2 depicts a combination of interface surfaces, for example, a mesh (20) and a macro-geometries is combined to tailor an interface of preferred strength.

A surface with geometries such as recess, undercuts (80), grooves (see FIGS. 4-5) or like that allowed the polymeric material to penetrate and consolidate taking the shape of that surface such that the mechanical interlocking achieved allows for a strong interface.

FIG. 3 shows a polyethylene (120), which is direct compression molded to a metallic mesh counterface (20) and/or a metal back metal shell (100) with a mechanical interlock, thereby forming a hybrid component The hybrid component was irradiated using ionizing radiation to a desired dose level, for example, about 25 kGy to about 1000 kGy, preferably between about 50 kGy and about 100 kGy. Irradiation crosslinked the polymer, as well as sterilized the interface that is in close contact between the polyethylene and counterface. During the process of irradiation, residual free radicals were generated, which may compromise the long-term oxidative stability of the polymer and in vivo device performance. Therefore, a melting step at this point in the fabrication is used to quench the residual free radicals. Because the resin powder was consolidated into the shape of the interface, which sets the shape memory of the polymer, the polyethylene did not separate from the counterface.

### Example 2

### Melting of Interlocked Interface in a Non-Oxidizing Medium

A medical device, for example, manufactured following a process as described in the examples 1 or 2, was immersed in a non-oxidizing medium such as inert gas or inert fluid. The medium was heated to above the melting point of the irradiated UHMWPE (about >137° C.) to eliminate the crystalline matter and allowed for the recombination/elimination of the residual free radicals. Because the shape memory of the compression molded polymer is set at the mechanically interlocked interface and that memory is strengthened by the crosslinking step, there was no significant separation at the interface between the polyethylene and the counterface.

### Example 3

### Post-Melting Machining and Sterilization

The interface between the metal and the polymer becomes sterile during the high dose irradiation used to manufacture a hybrid component following a process as described above in examples 1 and 2. When there is a substantial oxidation of the outside surface of the polyethylene, one can further machine this surface to remove the oxidized surface layer. This requires to oversize the outside surfaces (near net-shape) or not even forming the outside surface geometry during the compression molding process. In the case a post-melting machining step, the melting step also can be carried out in an inert gas.

At this point in the fabrication of the device, because the interface is sterile but the rest of the component is not, the implant is sterilized with ethylene oxide, gas plasma, or the other gas.

### Example 4

### Preparation of a Polyethylene/Mesh Assembly by High Temperature Compression of a Consolidated Polyethylene Cylinder to a Metallic Mesh:

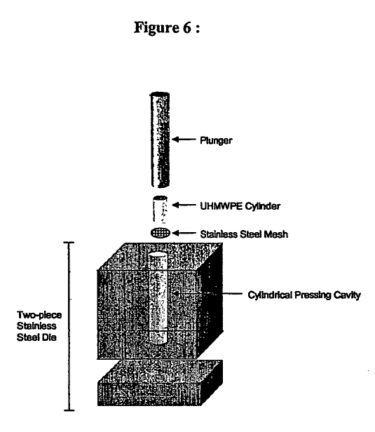

A custom-built, two-piece stainless steel die (FIG. 6), with central cylindrical cavity (diam.=11 mm) and accompanying stainless steel plunger (diam.=10.5 mm) was used in the compression molding experiments.

A SS304 woven wire mesh (Southwestern Wire Cloth, Tulsa, Okla.) with 20 mesh per inch, 0.016″ wire diameter, 0.034″ opening width, 46.2% open area was used along with a machined cylindrical pin of GUR 1050 ultra-high molecular weight (Perplas Ltd., Bacup, UK) with a diameter of 9 mm and height of 13 mm were used in the compression experiments.

First, a section of mesh was cut to drop into the central cylindrical cavity of the die. Next, the UHWMPE pin was placed inside the cavity. The plunger was added atop of the pin/mesh tandem, and the entire die assembly was heated to 160° C. under vacuum (Lindberg/Blue Vacuum Oven, Asheville, N.C.), allowing the UHMWPE pin to fully melt before pressing. Following heating, approximately 3,500 lbs of load was applied to the plunger using a Carver Hydraulic Press (Unit #3912, Wabash, Ind.). Once this maximum load level was reached, the die assembly was held under load for approximately 20 minutes, allowing load to decay as consolidation and/or creep occurred.

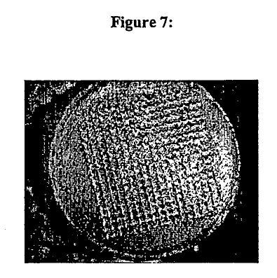

Five pin/mesh assemblies were fabricated. A typical example showing the mesh incorporated into the UHMWPE pin is displayed in FIG. 7.

### Example 5

### Compression Molding of Polyethylene Resin Powder to a Metallic Mesh

A custom-built, two-piece stainless steel die (FIG. 6), with central cylindrical cavity (diam.=11 mm) and accompanying stainless steel plunger (diam.=10.5 mm) was used in the compression molding experiments.

A SS304 woven wire mesh (Southwestern Wire Cloth, Tulsa, Okla.) with 60 mesh per inch, 0.0075″ wire diameter, 0.0092″ opening width, and 30.5% open area was used along with GUR 1050 ultra-high molecular weight virgin resin (Perplas Ltd., Bacup, ULK) flakes were used in the consolidation experiments.

First, a section of mesh was cut to drop into the central cylindrical cavity of the die. Next, the cavity was densely packed with UHMWPE resin powder. The plunger was added atop of the resin/mesh tandem, and the entire die assembly was heated to 210° C. under vacuum (Lindberg/Blue Vacuum Oven, Asheville, N.C.), allowing samples to fully melt before pressing. Following heating approximately 3,500 lbs of load was applied to the plunger using a Carver Hydraulic Press {Unit #3912, Wabash, Ind.). Once this maximum load level was reached, the die assembly was held under load for approximately 20 minutes, allowing load to decay as consolidation and/or creep occurred.

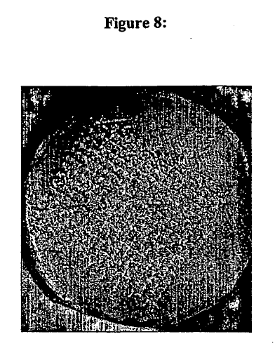

Five compression molded resin/mesh assemblies were fabricated. A typical example showing the mesh incorporated into polyethylene is displayed in FIG. 8.

### Example 6

### Fabrication of a Crosslinked UHMWPE Incorporated into a Metal-Mesh Backing with a Sterile Interface

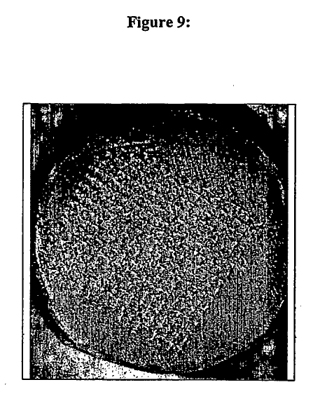

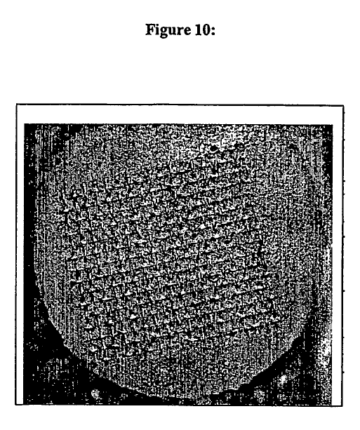

Five of the UHMWPE resin/mesh assemblies described in Example 5 were packaged in a metallized foil pouch in vacuum and subjected to 100 kGy gamma irradiation under vacuum (Steris-Isomedix, Northborough, Mass.). The faces of the UHMWPE resin/mesh assemblies that incorporated the mesh were photographed after irradiation. The UHMWPE resin/mesh assemblies were then heated to 160° C. in a vacuum to eliminate the residual free radicals generated by the gamma irradiation. The faces containing the mesh were photographed again following the melting. In all five samples the mesh was retained in the consolidated UHMWPE following the melting. In the present example, the shape memory of the UHMWPE was set to a geometry that included the mesh unlike what was observed in Example 4, where the shape memory recovery of the original dimensions of the pin led to the dissociation of the mesh. FIGS. 9-10 show examples of the retention of the mesh following post-irradiation melting.

Therefore, the polyethylene resin needs to be consolidated to a original shape memory that also includes the metallic structure of interest to avoid dissociation following post-irradiation melting.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A method of making a medical implant containing crosslinked polyethylene that is in contact with another piece, thereby forming an interface, wherein the method comprises:
a) compression molding of polyethylene to another piece, thereby forming an interlocked hybrid material;
b) irradiating the hybrid material by ionizing radiation; and
c) reducing free radicals in the crosslinked polyethylene by heating the hybrid material above the melting point of the crosslinked polyethylene.

2. The method according to claim 1, wherein the polyethylene comprises polyethylene resin powder, flakes, or particles, and wherein the polyethylene is compression molded to a metallic back.

3. The method according to claim 1, wherein the metallic back is shaped to serve as a fixation interface with the bone, through either bony growth or by bone cement.

4. The method according to claim 3, wherein the shapes are in the form of acetabular liner, tibial tray for total or unicompartmental knee implants, patella tray, glenoid component, ankle, elbow or finger component.

5. The method according to claim 1, wherein the irradiation is carried out in an atmosphere containing between about 1% and about 22% oxygen.

6. The method according to claim 1, wherein the irradiation is carried out in an inert atmosphere, wherein the inert atmosphere contains gas selected from the group consisting of nitrogen, argon, helium, neon, or the like, or a combination thereof.

7-11. (canceled)

12. The method according to claim 1, wherein the radiation dose is between about 25 and about 1000 kGy.

13-14. (canceled)

15. The method according to claim 1, wherein the piece is a metallic or a non metallic back, a ceramic, a tibial tray, a patella tray, or an acetabular shell.

16. The method of claim 1, wherein the piece comprises a metallic or a non-metallic mesh, an undercut, a recess or a combination thereof.

17-42. (canceled)

43. A method of forming and sterilizing a medical implant containing crosslinked polyethylene that is in contact with another piece, thereby forming an interface, wherein the method comprises the steps of:
a) compression molding of polyethylene to another piece, thereby forming an interlocked hybrid material;
b) irradiating the hybrid material by ionizing radiation;
c) reducing free radicals in the crosslinked polyethylene by heating the hybrid material above the melting point of the crosslinked polyethylene; and
d) sterilizing the medical implant with a gas.

44-46. (canceled)

47. The method according to claim 43, wherein the heating is carried out in an atmosphere containing between about 1% and about 22% oxygen.

48. The method according to claim 43, wherein the heating is carried out in an inert atmosphere, wherein the inert atmosphere contains gas selected from the group consisting of nitrogen, argon, helium, neon, or the like, or a combination thereof.

49-58. (canceled)

59. A medical implant containing crosslinked polyethylene that is in contact with another piece, thereby forming an interface, obtainable by:
a) compression molding of polyethylene to another piece, thereby forming an interlocked hybrid material;
b) irradiating the hybrid material by ionizing radiation; and
c) reducing free radicals in the crosslinked polyethylene by heating the hybrid material above the melting point of the crosslinked polyethylene.

60. The medical implant of claim 59, wherein the polyethylene is in contact with another piece, thereby forming an interlocking interface.

61. The medical implant of claim 59, wherein the interface is substantially sterile.

62-139. (canceled)

140. An acetabular assembly comprising:
a) polyethylene compression molded to another piece, thereby forming an interlocked hybrid component;
b) a substantially sterile interface; and
c) a metallic back.

141. The assembly of claim 140, wherein the piece comprising a metallic mesh, a non-metallic mesh, an undercut, a recess, or a combination thereof.

142. The assembly of claim 140, wherein the polyethylene comprises powder, flakes, or particles, and wherein the polyethylene is compression molded to a counterface.

143. The assembly of claim 142, wherein the counterface is metallic back, a metallic mesh, a tibial tray, a patella tray, or an acetabular shell.

144. The assembly of claim 142, wherein the counterface is shaped to serve as a fixation interface with the bone, through either bony growth or by bone cement.

145. The assembly of claim 144, wherein the shapes are in the form of acetabular liner, tibial tray for total or unicompartmental knee implants, patella tray, glenoid component, ankle, elbow or finger component.

146. The assembly of claim 140, wherein the polyethylene is crosslinked by ionizing radiation.

147-173. (canceled)

174. A medical implant comprising crosslinked polyethylene having substantially no detectable free radicals; and a sterile interlocking interface.

175. The implant of claim 174, wherein the polyethylene is in contact with another piece, thereby forming an interface.

176. The implant of claim 174, wherein the polyethylene is compression molded to another piece, thereby forming a mechanically interlocked hybrid material.

177-185. (canceled)
